SEQUENCE LISTING (16 sequences)

SEQ ID NO: 1 - DNA, 21 bp, Artificial Sequence
  Description of Artificial Sequence Synthetic oligonucleotide
gacaacagacaaatcaccatt

SEQ ID NO: 2 - DNA, 19 bp, Artificial Sequence
  Description of Artificial Sequence Synthetic oligonucleotide
gcccttcactactgttgac

SEQ ID NO: 3 - DNA, 17 bp, Artificial Sequence
  Description of Artificial Sequence Synthetic primer
gttggcaacctcatcgt

SEQ ID NO: 4 - DNA, 18 bp, Artificial Sequence
  Description of Artificial Sequence Synthetic primer
aattctgcagtgcctggt

SEQ ID NO: 5 - DNA, 24 bp, Artificial Sequence
  Description of Artificial Sequence Synthetic primer
gaactgatagcaagaaacacctgg

SEQ ID NO: 6 - DNA, 22 bp, Artificial Sequence
  Description of Artificial Sequence Synthetic primer
gtatcctggaacttggactttt

SEQ ID NO: 7 - DNA, 19 bp, Artificial Sequence
  Description of Artificial Sequence Synthetic primer
tccatgtatggcgtgaacc

SEQ ID NO: 8 - DNA, 20 bp, Artificial Sequence
  Description of Artificial Sequence Synthetic primer
cttcgaagatgtccagcacc

SEQ ID NO: 9 - DNA, 20 bp, Artificial Sequence
  Description of Artificial Sequence Synthetic primer
tactctggcaatgcctatgg

SEQ ID NO: 10 - DNA, 19 bp, Artificial Sequence
  Description of Artificial Sequence Synthetic primer
tacagatggtcaaaggccg

SEQ ID NO: 11 - DNA, 20 bp, Artificial Sequence
  Description of Artificial Sequence Synthetic primer
tctggcccaccaactttggg

SEQ ID NO: 12 - DNA, 20 bp, Artificial Sequence
  Description of Artificial Sequence Synthetic primer
cttcacaagcatgaactcca

SEQ ID NO: 13 - DNA, 18 bp, Artificial Sequence
  Description of Artificial Sequence Synthetic primer
aacctgggcaacatgagc

SEQ ID NO: 14 - DNA, 19 bp, Artificial Sequence
  Description of Artificial Sequence Synthetic primer
gtcgctccagctcgtacac

SEQ ID NO: 15 - DNA, 20 bp, Artificial Sequence
  Description of Artificial Sequence Synthetic primer
accacagtccatgccatcac

SEQ ID NO: 16 - DNA, 20 bp, Artificial Sequence
  Description of Artificial Sequence Synthetic primer
tccaccaccctgttgctgta

(19) United States
(12) Patent Application Publication
(10) Pub. No.: US 2008/0167532 A1
(43) Pub. Date: Jul. 10, 2008

(54) ASSESSING CANCER TREATMENT RESPONSIVENESS

(75) Inventors: Robert C. Smallridge, Ponte Vedra, FL (US); John A. Copland, Ponte Vedra Beach, FL (US)

Correspondence Address: FISH & RICHARDSON P.C., PO BOX 1022, MINNEAPOLIS, MN, 55440-1022, US

(73) Assignee: Mayo Foundation for Medical Education and Research, Rochester, MN (US)

(21) Appl. No.: 11/872,690

(22) Filed: Oct. 15, 2007

Related U.S. Application Data
(60) Provisional application No. 60/851,388, filed on Oct. 13, 2006.

(51) Int. Cl. A61B 5/00

(52) U.S. Cl. 600/300

(57) ABSTRACT

This document provides methods and materials related to assessing the effectiveness of cancer treatments in mammals. For example, this document provides nucleic acids and polypeptides that can be analyzed to assess the effectiveness of a treatment (e.g., treatment with a PPARγ agonist) for cancer (e.g., anaplastic thyroid carcinoma) in mammals.

### CROSS-REFERENCE TO RELATED APPLICATIONS

The present application claims the benefit of the filing date of U.S. Provisional Application No. 60/851,388, which was filed on Oct. 13, 2006. The disclosure of the prior application is considered part of (and is incorporated by reference in) the disclosure of this application.

### STATEMENT AS TO FEDERALLY SPONSORED RESEARCH

This invention was made with government support under CA15083-31DJ awarded by NIH. The government has certain rights in the invention.

### BACKGROUND

1. Technical Field

This document relates to methods and materials involved in assessing the effectiveness of cancer treatments in mammals. For example, this document provides methods and materials for determining whether a treatment (e.g., treatment with a PPARγ agonist) is effective against cancer (e.g., anaplastic thyroid carcinoma) in mammals.

2. Background Information

Peroxisome proliferator activated receptors (PPARs) are both hormone receptors and transcription factors found in the nucleus of many cells. PPARs therefore have two binding sites, one site for ligands (such as fatty acids, hormones, and specific diabetic drugs) and one site for DNA. There are three known subtypes of PPAR, alpha (α), delta (δ), and gamma (γ). The latter subtype, PPARγ is abundant in adipose tissue and plays a key role in fat cell differentiation. PPARγ is activated upon binding to a PPARγ agonist. Activated PPARγ forms a heterodimer with retinoid X receptor (RXR). The heterodimer binds to regions of DNA that regulate transcription of PPARγ target genes, and induces transcription of PPARγ regulated genes in concert with coactivators (Gearing et al, PNAS, 90:1440-1444 (1993); Lehrke and Lazar, Cell, 123:993-999 (2005); and Li et al., Critical Reviews in Clinical Laboratory Sciences, 43:183-202 (2006)).

### SUMMARY

This document provides methods and materials related to assessing the effectiveness of cancer treatments mammals (e.g., humans). For example, this document provides arrays for detecting polypeptides or nucleic acids that can be used to assess the effectiveness of a treatment (e.g., treatment with a PPARγ agonist) for cancer (e.g., anaplastic thyroid carcinoma) in mammals such as humans. Such arrays can allow the effectiveness of a cancer treatment to be assessed in mammals based on differences in the levels of polypeptides or nucleic acids in biological samples from mammals having cancer and having received the treatment, as compared to the corresponding levels in biological samples from mammals having cancer and not having received the treatment.

This document is based, in part, on the discovery of nucleic acid sequences that are differentially expressed between anaplastic thyroid carcinoma (ATC) cells treated with the PPARγ agonist RS5444, and ATC cells not treated with the PPARγ agonist RS5444. Such nucleic acids, as well as polypeptides encoded by such nucleic acids, can be analyzed to assess the effectiveness of cancer treatment (e.g., treatment with a PPARγ agonist) in mammals. Analysis of such nucleic acids, or polypeptides encoded by such nucleic acids, can allow the effectiveness of cancer treatment to be assessed in a mammal based on a difference in the level of one or more of the nucleic acids or polypeptides in a biological sample (e.g., a biopsy specimen) from the mammal having cancer and having received the treatment, as compared to the average level observed in corresponding samples from mammals having cancer and not having received the treatment. For example, a difference in the level of one or more of the nucleic acids listed in Table 1, or one or more polypeptides encoded by the nucleic acids listed in Table 1, in a sample from a mammal having cancer and having received a treatment for the cancer, as compared to the average level of the one or more nucleic acids, or one or more polypeptides encoded by the nucleic acids, in corresponding samples from mammals having cancer and not having received the treatment for the cancer, can indicate that the treatment is effective. In some cases, a difference in the level of one or more than one of a BMP2, MMP14, ANGPTL4, and ITGA5 polypeptide in blood from a mammal having cancer and having received a cancer treatment, as compared to the average level of the corresponding one or more than one polypeptide in blood from mammals having cancer and not having received the cancer treatment, can indicate that the treatment is effective.

In general, one aspect of this document features a method for assessing the effectiveness of a cancer treatment in a mammal. The method comprises, or consists essentially of, (a) determining whether or not the mammal has a therapeutic response profile, wherein the mammal has received the treatment, and (b) classifying the treatment as effective if the mammal has the therapeutic response profile and classifying the as not being effective if the mammal does not have the therapeutic response profile. The mammal can be a human. The treatment can comprise treatment with a PPARγ agonist. The PPARγ agonist can be rosiglitazone or troglitazone. The profile can be determined in thyroid tissue. The thyroid tissue can be biopsy tissue. The biopsy tissue can be obtained within 24 hours of the treatment. The profile can be determined using PCR or a nucleic acid array. The profile can be determined using immunohistochemistry or an array for detecting polypeptides.

In another aspect, this document features a method for assessing the effectiveness of a cancer treatment in a mammal. The method comprises, or consists essentially of, (a) determining whether or not a mammal having cancer and having received a cancer treatment comprises RhoB nucleic acid or polypeptide at a level that differs from the average level in mammals having cancer and not having received a cancer treatment, and (b) classifying the treatment as effective if the level differs from the average level and classifying the treatment as ineffective if the level does not differ from the average level.

In another aspect, this document features a method for assessing the effectiveness of a cancer treatment in a mammal. The method comprises, or consists essentially of, (a) determining whether or not a mammal having cancer and having received a cancer treatment comprises a BMP2, MMP14, ANGPTL4, or ITGA5 nucleic acid or polypeptide at a level that differs from the average level in mammals having cancer and not having received a cancer treatment, and (b) classifying the treatment as effective if the level differs from the average level and classifying the treatment as ineffective if the level does not differ from the average level. The level can be determined in blood.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF THE DRAWINGS

FIG. 1 is a schematic diagram illustrating the sequence of events that occurs after contacting human ATC cells with a PPARγ agonist, including upregulation of RhoB and p21^{WAF1/CIP1} polypeptides, down-regulation of cyclin E, and inhibition of tumor growth.

FIG. 2 contains photomicrographs of formalin-fixed, paraffin-embedded surgical tissues stained with H&E and analyzed by immunohistochemistry using antibodies specific for PPARγ and RXRα. Black arrows point to cells that stained positive for PPARγ expression. White arrows indicate no staining; the cells appear blue due to hemotoxylin staining.

FIG. 3A is a graph plotting firefly luciferase activity normalized to renilla firefly activity (RLU) and as mean values ±S.D. in DRO cells that were transfected with a PPRE3-tk-luc reporter and treated with the indicated doses of RS5444, rosiglitazone, or troglitazone for 24 hours. FIG. 3B is a graph plotting the mean cell number ±S.D. in DRO cell cultures treated with the indicated concentrations of RS5444, rosiglitazone, or troglitazone every 48 hours for six days. IC50 values were calculated to be about 0.8 nM for RS5444, about 75 nM for rosiglitazone, and about 1.4 μM for troglitazone. FIG. 3C is a graph plotting the number of colonies of DRO cells grown in soft agar versus the dose of RS5444 used to treat the cells. *p<0.01 for comparisons between treated and control cultures. FIG. 3D is a graph plotting tumor volume versus days of treatment with the indicated amounts of RS5444 in the diet of athymic nude mice. RS5444 was added to the rodent diet one week prior to implanting DRO cells.

FIG. 4A contains photomicrographs of normal and cancerous lung and thyroid (ATC) tissues analyzed by immunohistochemistry using a polyclonal anti-RhoB antibody.

FIG. 5 contains graphs plotting fold change in RhoB or p21^{WAF1/CIP1} RNA expression at various time points in DRO cells treated with 10 nM RS5444 or 0.1% DMSO. The data were normalized to DMSO treated controls for each time point measured.

FIG. 6A is a Western blot analyzing expression of RhoB, RhoA, and β-actin polypeptides in DRO cells treated with vehicle control (0.1% DMSO), 10 nM RS5444, 100 nM rosiglitazone, 1 μM troglitazone, GW9662 (PPARγ antagonist), or GW9662 plus 10 nM RS5444. FIG. 6B is a Western blot analyzing expression of RhoB, p21^{WAF1/CIP1} (p21), Cyclin E, and β-actin polypeptides in DRO cells treated at the indicated times with DMSO vehicle control (−) or 10 nM RS5444 (+). FIG. 6C is a Western blot analyzing expression of RhoB and p21^{WAF1/CIP1} polypeptides in DRO and ARO tumor tissues removed from athymic nude mice following 35 days of treatment with vehicle control (−) or 0.025% RS5444 in the diet.

FIG. 7 is a Western blot analyzing expression of RhoB, p21^{WAF1/CIP1}, and β-actin polypeptides in DRO cells that were transfected with siRNA against RhoB or scrambled control siRNA, and treated with 10 nM RS5444 for 24 hours.

FIG. 8 contains a series of fluorescence photomicrographs of DRO, KTC-2, KTC-3 cells treated for 24 hours with 10 nM RS5444 and analyzed by immunocytochemistry using fluorescently labeled antibodies to detect RhoB (FITC, green) and p21 (Texas red). The immunofluorescence photographs were taken at 60× magnification.

FIG. 9A is a Western blot analyzing expression of PPARγ, RXRα, and βactin polypeptides in DRO cells transfected with scrambled (−) siRNA or siRNA targeted to PPARγ (+). FIG. 9B is a graph plotting relative light units (RLU) in cells that were transfected with siRNA for 72 hours followed by transfection with PPRE3-tk-luc reporter and treatment with 10 nM RS5444 for 24 hours. PPRE3-tk-luc transfected cells (24 h) that were treated with 10 μM GW9662 followed by 10 nM RS5444 one hour later demonstrated complete loss of RS5444 inducible luciferase activity. Cotransfection with renilla luciferase allowed for normalization to relative light units (RLU). FIG. 9C is a graph plotting RLU in PPRE3-tk-luc transfected DRO cells pretreated for five minutes with 10 μM GW9662 prior to being treated with 10 nM RS5444, 1 μM rosiglitazone, or 10 μM troglitazone. FIG. 9D is a graph plotting cell number/well for cultures of DRO cells pretreated for one hour with 10 μM GW9662 prior to being treated with 10 nM RS5444, 1 μM rosiglitazone, or 10 μM troglitazone every 48 hours over six days. The data are plotted as mean values ±S.D. The * indicates p<0.01 for comparisons between treated and control cultures, and ** indicates p<0.05 for comparisons between cultures treated with GW9662/agonist or GW9662 alone.

FIG. 10A contains photomicrographs of cells that were transfected with scrambled siRNA or siRNA targeted to PPARγ, and mock-treated or treated with RS5444 or GW9662 and RS5444, and analyzed for expression of p21^{WAF1/CIP1}. FIG. 10B is a Western blot analyzing expression of p21^{WAF1/CIP1}, p27, PPARγ, and RXRα polypeptides in cells that were transfected with control siRNA or siRNA targeted to p21^{WAF1/CIP1}, and that were treated with RS5444. FIG. 10C is a graph plotting cell number/well in cell cultures that were transfected with control siRNA or siRNA targeted to p21^{WAF1/CIP1}, and that were mock-treated or treated with 10 nM RS5444.

FIG. 11A is a photograph of a western blot for PPAR-γ expression/silencing in the four indicated ATC cell lines stably infected with either non-target or PPAR-γ shRNA lentivirus. This figure demonstrates that shRNA PPAR-γ silences PPAR-γ.

FIG. 11B is a graph plotting firefly luciferase activity (normalized to renilla firefly activity (RLU) and graphed as RLU as compared to non-target control ±S.D.) in the indicated ATC cell lines transfected with a PPRE3-tk-luc reporter and stably expressing either non-target or PPAR-γ shRNA and treated with or without 10 nM RS5444 for 24 hours. Silencing PPAR-γ results in complete blockade of 10 nM RS5444 induction of PPAR-γ mediated transcriptional activity (FIG. 11B). FIG. 11C is a bar graph plotting cell proliferation as percent of non-target control ±S.D for the indicated ATC cell lines stably expressing either non-target or PPAR-γ shRNA and treated with or without 10 nM RS 5444 over 6 days of treatment. The cells were plated for six days as described in Example 6. The asterisk indicates statistical difference (p<0.05) when compared to control plus 10 nM RS5444. Silencing PPAR-γ resulted in the loss in the ability of RS5444 to inhibit cell proliferation in four ATC cell lines. This indicates that RS5444 mediates its growth inhibitory activity via PPAR-γ.

FIG. 12A contains microscopic images of KTC2 and KTC3 cell lines treated with and without 10 nM RS5444 for 24 hours at 40× magnification in which cell shape is altered by RS5444 causing cells to appear stressed. FIG. 12B is a table listing real time PCR results of RhoB, RhoA, RhoC, Rac1, and cdc42 in four ATC cell lines treated with 10 nM RS5444 in which RhoB is identified to be upregulated at the mRNA levels suggesting that RS5444 transcriptionally regulates RhoB. These results also suggest that the morphological changes are likely due to RhoB expression. FIG. 12C is a photograph of PPARγ, RhoA, and RhoB immunoblotting of DRO cells transfected for 72 hours with scrambled or PPARγ siRNA and treated with (+) or without (−) 10 nM RS5444. These results demonstrate the lack of RhoB upregulation in the absence of PPAR-γ despite the presence of 10 nM RS5444. FIG. 12D is a photograph of a RhoA and RhoB western blot analysis of DRO cells treated with 10 nM RS5444, 100 nM rosiglitazone, 1 μM troglitazone, and 10 μM GW9662 for 24 hours. In lane 5, the PPARγ antagonist GW9662 was added 5 minutes prior to RS5444. RhoB polypeptide expression was induced most strongly by RS5444 and was abolished by GW9662, with no change in RhoA polypeptide expression. FIG. 12E is a photograph of a RhoB western blot analysis of the indicated ATC tumors from mice following 4 weeks of 0.025% RS5444 treatment (+) as compared to the vehicle control (−). Results from three animals of each group are shown where RhoB polypeptide is elevated in the tumor of animals treated chronically with 0.025% RS5444 in the diet.

FIG. 13A is a photograph of a RhoB western blot analysis of four ATC cell lines verifying that RhoB expression remained silenced even in the presence of RS5444. Cells were transfected for 24 hours with scrambled (scr) and RhoB siRNA and treated with 10 nM RS5444. FIG. 13B is a graph plotting cellular proliferation examined by transfecting ATC cell lines with RhoB or scrambled siRNA for 24 hours and at 48 hour intervals. Cells were treated with the indicated compounds every 48 hours for 6 days beginning 24 hour after the first RNAi treatment. Cell numbers were counted and data plotted as percent of scrambled control ±S.D. The asterisk indicates p<0.05 when compared to scrambled plus RS5444. The data indicate in four ATC cell lines that RS5444 growth inhibition is dependent upon RhoB polypeptide expression.

FIG. 14A is a graph plotting real time PCR levels of RhoB mRNA in DRO cells upon exposure to DMSO or 10 nM RS5444. These results demonstrate RhoB mRNA induction as early as 2 hours. FIG. 14B is a graph plotting real time PCR levels of p21 mRNA in DRO cells upon exposure to DMSO or 10 nM RS5444. These results demonstrate p21 mRNA induction by 4 hours. This data is different from that of FIG. 5B. The data presented in FIG. 14B has been repeated multiple times by various individuals in the laboratory whereas the data presented in FIG. 5B was performed by a single individual. In each case, real time PCR was performed in triplicate, and data normalized to 18S. Data were plotted as means ±SD with n=4. The asterisk indicates p<0.05 when compared to DMSO alone. FIG. 14C is a photograph of RhoA, RhoB, and p21 immunoblotting of DRO cell extracts treated with (+) or without (−) 10 nM RS5444 for the indicated time. These results demonstrate the upregulation of both RhoB and p21 polypeptides with no change in RhoA polypeptide levels.

FIG. 15A is a bar graph plotting mRNA expression levels (via real time PCR) of RhoB and p21 mRNA in DRO cells after transfection for 24 hours with scrambled and RhoB siRNA and then treated with 10 nM RS5444. These results demonstrate p21 mRNA's dependence upon RhoB induction when stimulated by RS5444. FIG. 15B is a bar graph plotting mRNA expression levels (via real time PCR) of RhoB and p21 mRNA levels in DRO cells after transfection for 24 hours with pcDNA3.1 and dominant negative (DN) RhoB and then treated with RS5444 for 24 hours. These results demonstrate p21 mRNA's dependence upon RhoB induction through RS5444/PPAR-γ. FIG. 15C is a photograph of western blot results from DRO cells transfected for 24 hours with scrambled and RhoB siRNA and then treated with 10 nM RS5444. These results demonstrate the requirement of RS5444-induced RhoB upregulation for RS5444-induced p21 upregulation. FIG. 15D is a photograph of western blot results from DRO cells transfected for 24 hours with scrambled and p2I siRNA and treated with RS5444. These results demonstrate that RhoB's effects are upstream of p21 since RhoB levels remained unchanged in the presence of RS5444 in response to p21 silencing.

FIG. 16A is a photograph of a RhoB and p21 western blot analysis of DRO cells treated for 24 hours with 10 nM RS5444, 1 ng/mL FK-228, and 10 μM FTI-277. These results demonstrate upregulation of both RhoB and p21 expression. FIG. 16B is a bar graph plotting cellular proliferation of DRO cells transfected with either RhoB or scrambled siRNA for 24 hours and at 48-hour intervals. Cells were treated with the indicated compounds every 48 hours for 6 days beginning 24 hours after the first RNAi treatment. Cell numbers were counted, and data plotted as total cell number ±S.D. The asterisk indicates p<0.05 when compared to scrambled plus treatment. The results demonstrate that FK228 and FTI-277 are dependent upon the upregulation of RhoB polypeptide to inhibit cell proliferation. FIG. 16C is a diagram of a model depicting that RhoB modulators such as PPAR-γ agonists, HDAC inhibitors, and farnesyl transferase inhibitors induce RhoB expression in order to induce p21 expression, which in turn inhibits cellular proliferation.

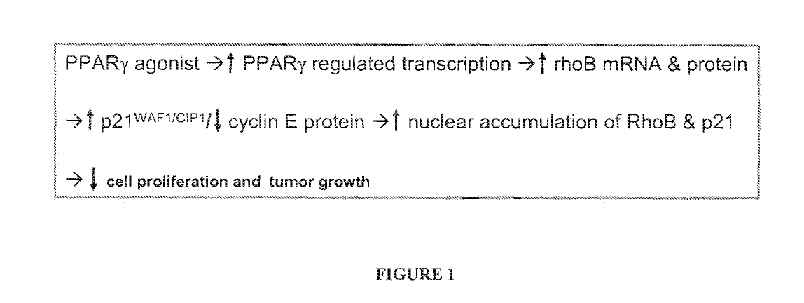

### DETAILED DESCRIPTION

This document provides methods and materials related to assessing the effectiveness of cancer treatments in mammals. For example, this document provides methods and materials for determining whether or not a biological sample (e.g., a thyroid tissue sample) from a mammal having cancer (e.g., ATC) and having received a treatment (e.g., a PPARγ agonist) contains a difference in the level of one or more than one therapeutic response biomarker, as compared to the level of the one or more than one therapeutic response biomarker in a sample obtained from the mammal prior to receiving the treatment, or as compared to the average level in samples from other mammals having the same type of cancer and not having received the treatment. A therapeutic response biomarker can be a RhoB, BMP2, ANGPTL4, MMP14, HR, ITGA5, IL1B, ADRB1, LMAN1, PPARγ, RXRα, p21^{WAF1/CIP1}, or cyclin E polypeptide. In some cases, a therapeutic response biomarker can be a RhoB, BMP2, ANGPTL4, MMP14, HR, ITGA5, IL1B, ADRB1, LMAN1, PPARγ, RXRα, or cyclin E nucleic acid. As described herein, a level of a therapeutic response biomarker in a sample from a mammal having cancer and having received a cancer treatment, that differs from the level of that therapeutic response biomarker in a sample from the mammal obtained prior to treatment of the mammal, can indicate that the treatment is effective. In some cases, a level of a therapeutic response biomarker (e.g., RhoB) in a sample from a mammal having cancer and having received a cancer treatment, that differs from the average level of that therapeutic response biomarker (e.g., RhoB) in samples from mammals having cancer and not having received cancer treatment (or mammals having cancer and having received a cancer treatment that was unsuccessful), can indicate that the treatment is effective. In some cases, a level of one or more than one of a BMP2, MMP14, ANGPTL4, and ITCA5 polypeptide in blood from a mammal having cancer and having received a cancer treatment, that differs from the level of the one or more than one polypeptide in blood from the mammal prior to treatment, or that differs from the average level of the one or more than one polypeptide in blood from mammals having cancer and not having received cancer treatment (or mammals having cancer and having received a cancer treatment that was unsuccessful), can indicate that the treatment is effective.

In some cases, a treatment can be classified as being effective if it is determined that a sample from a mammal having cancer and having received the treatment has a therapeutic response profile. For the purpose of this document, the term “therapeutic response profile” as used herein refers to a nucleic acid or polypeptide profile in a sample where one or more than one (e.g., one, two, three, four, five, six, seven, eight, nine, ten, eleven, or twelve) of a RhoB, BMP2, ANCPTL4, MMP14, HR, ITGA5, IL1B, ADRB1, LMAN1, PPARγ, RXRα, or cyclin E nucleic acid or polypeptide, or fragment thereof, in a sample from a mammal having cancer and having received a cancer treatment, differs from the corresponding level of the one or more than nucleic acid or polypeptide in a sample from the mammal prior to treatment, or that differs from the average level of the one or more than one nucleic acid or polypeptide in samples from mammals having cancer and not having received the treatment (or mammals having cancer and having received a cancer treatment that was unsuccessful). In some cases, the therapeutic response profile can be a profile in a sample where a majority of a RhoB, BMP2, ANCPTL4, MMP14, HR, ITCA5, IL1B, ADRB1, LMAN1, PPARγ, RXRα, or cyclin E nucleic acid or polypeptide is present at level that differs from the average level or the level prior to treatment.

To assess the effectiveness of a cancer treatment (e.g., RS5444, rosiglitazone, troglitazone, an HMG-CoA reductase inhibitor, an HDAC inhibitor, or a farnesyl transferase inhibitor) in a mammal, the level of one or more than one therapeutic response biomarker can be analyzed in a sample from the mammal. The level of a therapeutic response biomarker can be determined by measuring any therapeutic response biomarker including, without limitation, native, truncated, and mutant therapeutic response biomarkers, as well as any fragments thereof. Examples of therapeutic response biomarkers are listed in Table 1.

The level of a therapeutic response biomarker can be greater than or less than the average level observed in corresponding control samples (e.g., corresponding samples from mammals having cancer and not having received a cancer treatment). Typically, a therapeutic response biomarker can be classified as being present at a level that is greater than or less than the average level observed in control samples if the levels differ by at least 10, 20, 30, 40, 50, 60, 70, 80, 90, or more percent. In some cases, a nucleic acid or polypeptide can be classified as being present at a level that is greater than or less than the average level observed in control samples if the levels differ by greater than 1-fold (e.g., 1.5-fold, 2-fold, 3-fold, or more than 3-fold). Control samples typically are obtained from one or more mammals of the same species as the mammal being evaluated. Control samples can be obtained from mammals, such as humans, who have the same type of cancer as the cancer being treated, and who have not received a cancer treatment. In some cases, a control sample can be obtained from the mammal being treated for cancer, as long as the control sample is obtained prior to the treatment. Control samples can be obtained from any number of mammals. For example, control samples can be obtained from one or more mammals (e.g., 10, 20, 30, 40, 50, 75, 100, 200, 300, 400, 500, 1000, or more than 1000 mammals) from the same species as the mammal being evaluated.

It will be appreciated that levels from comparable samples are used when determining whether or not a particular level differs from an average level. For example, the average level of a therapeutic response biomarker present in ATC tissue from a random sampling of mammals (e.g., mammals not having received cancer treatment) may be X units/g of ATC tissue, while the average level of the therapeutic response biomarker present in blood may be Y units/volume of blood. In this case, the average level for the therapeutic response biomarker in ATC tissue would be X units/g of ATC tissue, and the average level for the therapeutic response biomarker in blood would be Y units/volume of blood. Thus, when determining whether or not the level of a therapeutic response biomarker in ATC tissue from a mammal having received cancer treatment differs from the average level of that therapeutic response biomarker in mammals not having received cancer treatment, the measured level would be compared to the average level for the therapeutic response biomarker in ATC tissue.

An elevated or reduced level of a therapeutic response biomarker can be any level provided that the level is greater or less than a corresponding level for that therapeutic response biomarker in a sample obtained from the mammal prior to receiving cancer treatment, or provided that the level is greater or less than the corresponding average level for that therapeutic response biomarker in multiple samples from mammals having cancer and not having received cancer treatment. For example, an elevated level of a therapeutic response biomarker can be 1.1, 1.2, 1.3, 1.4, 1.5, 1.6, 1.7, 1.8, 1.9, 2.0, 2.2, 2.4, 2.6, 2.8, 3, 3.2, 3.4, 3.6, 3.8, 4, 4.2, 4.4, 4.6, 4.8, 5, 6, 7, 8, 9, 10, 15, 20, or more times greater than the average level for that therapeutic response biomarker. In some cases, a reduced level of a therapeutic response biomarker can be 0.9, 0.8, 0.7, 0.6, 0.5, 0.4, 0.3, 0.2, or 0.1 times the average level for that therapeutic response biomarker. In addition, an average level can be any amount. For example, an average level for a therapeutic response biomarker can be zero. In this case, any level of the therapeutic response biomarker greater than zero would be an elevated level.

Any method can be used to determine the level of a therapeutic response biomarker present within a sample. For example, quantitative PCR, in situ hybridization, or microarray technology can be used to determine the level of a therapeutic response biomarker in a sample. In some cases, the level of a therapeutic response biomarker can be determined using polypeptide detection methods such as immunochemistry techniques. For example, antibodies specific for a therapeutic response biomarker can be used to determine the polypeptide level of the therapeutic response biomarker in a sample.

Any type of sample can be used to evaluate the level of a therapeutic response biomarker including, without limitation, thyroid tissue and blood. In addition, any method can be used to obtain a sample. For example, a thyroid tissue sample can be obtained by a tissue biopsy or following surgical resection. Once obtained, a sample can be processed prior to measuring the level of a therapeutic response biomarker. For example, a thyroid tissue sample can be processed to extract RNA from the sample. Once obtained, the RNA can be evaluated to determine the level of one or more than one therapeutic response biomarker present. In some embodiments, nucleic acids present within a sample can be amplified (e.g., linearly amplified) prior to determining the level of one or more than one therapeutic response biomarker (e.g., using array technology). In another example, a thyroid tissue sample can be frozen, and sections of the frozen tissue sample can be prepared on glass slides. The frozen tissue sections can be stored (e.g., at −80° C.) prior to analysis, or they can be analyzed immediately (e.g., by immunohistochemistry with an antibody specific for a therapeutic response biomarker).

Once the level of a therapeutic response biomarker in a sample from a mammal having received a cancer treatment is determined, then the level can be compared to an average level for that therapeutic response biomarker in control samples, or to the level of that therapeutic response biomarker in a sample from the mammal prior to being treated, and used to evaluate the effectiveness of the cancer treatment. A level of one or more than one therapeutic response biomarker in a sample from a mammal that differs from the corresponding one or more than one average level can indicate that the treatment is effective. In contrast, a level of one or more than one therapeutic response biomarker in a sample from a mammal that does not differ from the corresponding one or more than one average level can indicate that the treatment is not effective.

In some cases, the effectiveness of a treatment can be assessed based on the number of therapeutic response biomarkers having a different level in a sample from a mammal as compared to an average level or the level prior to treatment. The greater the number, the more effective is the treatment. In addition, the greater the differences between the levels of the therapeutic response biomarkers and the corresponding average levels, or the levels prior to treatment, the more effective is the treatment.

This document also provides methods and materials to assist medical or research professionals in determining whether or not a cancer therapy is effective. Medical professionals can be, for example, doctors, nurses, medical laboratory technologists, and pharmacists. Research professionals can be, for example, principle investigators, research technicians, postdoctoral trainees, and graduate students. A professional can be assisted by (1) determining the level of one or more than one therapeutic response biomarker in a sample, and (2) communicating information about that level to that professional.

Any method can be used to communicate information to another person (e.g., a professional). For example, information can be given directly or indirectly to a professional. In addition, any type of communication can be used to communicate the information. For example, mail, e-mail, telephone, and face-to-face interactions can be used. The information also can be communicated to a professional by making that information electronically available to the professional. For example, the information can be communicated to a professional by placing the information on a computer database such that the professional can access the information. In addition, the information can be communicated to a hospital, clinic, or research facility serving as an agent for the professional.

This document also provides nucleic acid arrays. The arrays provided herein can be two-dimensional arrays, and can contain at least two different nucleic acid molecules (e.g., at least three, at least five, at least ten, at least 20, at least 30, at least 40, at least 50, or at least 60 different nucleic acid molecules). Each nucleic acid molecule can have any length. For example, each nucleic acid molecule can be between 10 and 250 nucleotides (e.g., between 12 and 200, 14 and 175, 15 and 150, 16 and 125, 18 and 100, 20 and 75, or and 50 nucleotides) in length. In some cases, an array can contain one or more cDNA molecules encoding, for example, partial or entire polypeptides. In addition, each nucleic acid molecule can have any sequence. For example, the nucleic acid molecules of the arrays provided herein can contain sequences that are present within therapeutic response biomarkers.

In some cases, at least 25% (e.g., at least 30%, at least 40%, at least 50%, at least 60%, at least 75%, at least 80%, at least 90%, at least 95%, or 100%) of the nucleic acid molecules of an array provided herein contain a sequence that is (1) at least 10 nucleotides (e.g., at least 11, 12, 13, 14, 15, 16, 17, 18, 19, 20, 25, or more nucleotides) in length and (2) at least about 95 percent (e.g., at least about 96, 97, 98, 99, or 100) percent identical, over that length, to a sequence present within a therapeutic response biomarker. For example, an array can contain 60 nucleic acid molecules located in known positions, where each of the 60 nucleic acid molecules is 100 nucleotides in length while containing a sequence that is (1) 90 nucleotides is length, and (2) 100 percent identical, over that 90 nucleotide length, to a sequence of a therapeutic response biomarker. A nucleic acid molecule of an array provided herein can contain a sequence present within a therapeutic response biomarker where that sequence contains one or more (e.g., one, two, three, four, or more) mismatches.

The nucleic acid arrays provided herein can contain nucleic acid molecules attached to any suitable surface (e.g., plastic or glass). In addition, any appropriate method can be used to make a nucleic acid array. For example, spotting techniques and in situ synthesis techniques can be used to make nucleic acid arrays. Further, the methods disclosed in U.S. Pat. Nos. 5,744,305 and 5,143,854 can be used to make nucleic acid arrays.

This document also provides arrays for detecting polypeptides. The arrays provided herein can be two-dimensional arrays, and can contain at least two different polypeptides capable of detecting polypeptides, such as antibodies (e.g., at least three, at least five, at least ten, at least 20, at least 30, at least 40, at least 50, or at least 60 different polypeptides capable of detecting polypeptides). The arrays provided herein also can contain multiple copies of each of many different polypeptides. In addition, the arrays for detecting polypeptides provided herein can contain polypeptides attached to any suitable surface (e.g., plastic or glass).

A polypeptide capable of detecting a polypeptide can be naturally occurring, recombinant, or synthetic. The polypeptides immobilized on an array also can be antibodies. An antibody can be, without limitation, a polyclonal, monoclonal, human, humanized, chimeric, or single-chain antibody, or an antibody fragment having binding activity, such as a Fab fragment, F(ab′) fragment, Fd fragment, fragment produced by a Fab expression library, fragment comprising a VL or VH domain, or epitope binding fragment of any of the above. An antibody can be of any type, (e.g., IgG, IgM, IgD, IgA or IgY), class (e.g., IgG1, IgG4, or IgA2), or subclass. In addition, an antibody can be from any animal including birds and mammals. For example, an antibody can be a mouse, chicken, human, rabbit, sheep, or goat antibody. Such an antibody can be capable of binding specifically to a therapeutic response biomarker. The polypeptides immobilized on the array can be members of a family such as a receptor family, protease family, or an enzyme family.

Antibodies can be generated and purified using any suitable methods known in the art. For example, monoclonal antibodies can be prepared using hybridoma, recombinant, or phage display technology, or a combination of such techniques. In some cases, antibody fragments can be produced synthetically or recombinantly from a nucleic acid encoding the partial antibody sequence. In some cases, an antibody fragment can be enzymatically or chemically produced by fragmentation of an intact antibody. In addition, numerous antibodies are available commercially. An antibody directed against a therapeutic response biomarker can bind the polypeptide at an affinity of at least 10⁴ mol⁻¹ (e.g., at least 10⁵, 10⁶, 10⁷, 10⁸, 10⁹, 10¹⁰, 10¹¹, or 10¹² mol⁻¹).

Any method can be used to make an array for detecting polypeptides. For example, methods disclosed in U.S. Pat. No. 6,630,358 can be used to make arrays for detecting polypeptides. Arrays for detecting polypeptides can also be obtained commercially, such as from Panomics, Redwood City, Calif.

The invention will be farther described in the following examples, which do not limit the scope of the invention described in the claims.

### EXAMPLES

### Example 1

### PPARγ Agonists Inhibit Growth of ATC Cells

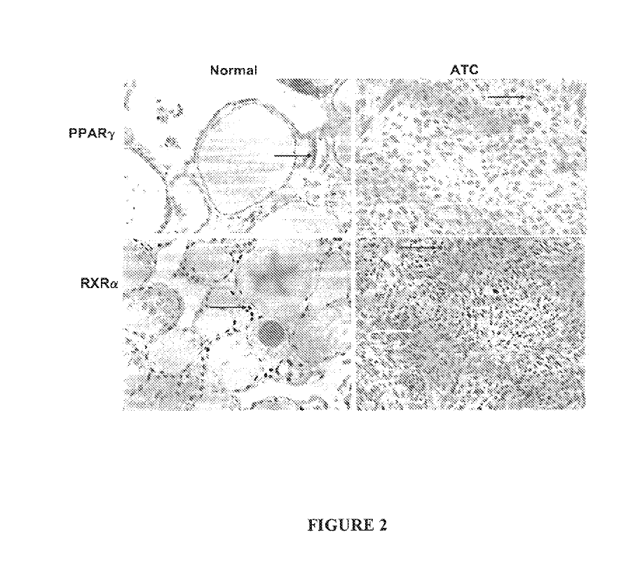

Expression of PPARγ RNA and polypeptide was measured in normal thyroid and anaplastic thyroid carcinoma (ATC) tissues from patient samples. Formalin fixed/paraffin embedded tissues were examined for PPARγ and RXRα polypeptide expression. Three ATC tissues were examined. Standard procedures were used with the E8 monoclonal primary antibody for PPARγ (Santa Cruz Biotechnology, Santa Cruz, Calif.; 1:40 dilution) and RXRα (Active Motif; 1:50 dilution). PPARγ and RXRα polypeptides were expressed in normal thyroid and ATC tissues. PPARγ was expressed in all three ATC tissues examined (FIG. 2). Brown staining for PPARγ was evident in the nucleus of normal and ATC tissues (FIG. 2, black arrows), while blue staining (hematoxylin) indicated a lack of PPARγ expression (FIG. 2, white arrows). Specificity of the antibody for PPARγ was established by performing immunocytochemistry (ICC) on RIE cells that do or do not express PPARγ.

PPARγ RNA expression was measured in six normal thyroid samples and six ATC tissue samples using real-time PCR. There was no difference in PPARγ mRNA expression between normal thyroid and ATC tissues (ΔCt Normal Avg +/−S.D. was 21.11±1.48, and ΔCt Tumor Avg +/−S.D. was 21.05±1.52; data normalized to 18s RNA).

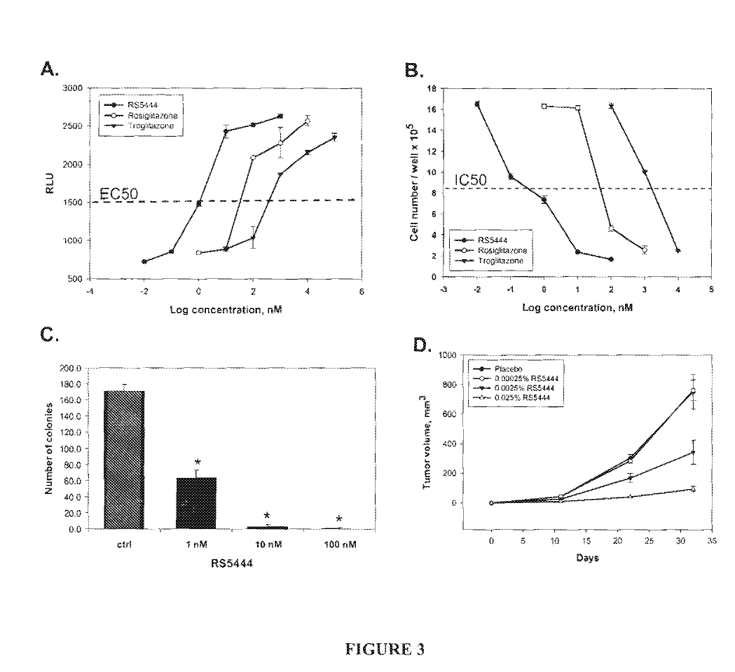

The effects of PPARγ agonists on DRO cells were investigated. DRO cells were transfected with a PPRE3-tk-luc reporter and treated with various doses of RS5444, rosiglitazone, or troglitazone for 24 hours to examine effective concentrations at which 50% maximal transcriptional response (EC50) occurs. Firefly luciferase activity was normalized to renilla firefly activity (RLU) and plotted as mean values ±S.D. (FIG. 3A). The EC50s were about 1 nM (RS5444), about 65 nM (rosiglitazone) and about 631 nM (troglitazone).

Concentrations of RS5444, rosiglitazone, and troglitazone that inhibited 50% of DRO cell proliferation were determined. Cells were treated every 48 hours over six days. Cell numbers were counted and data were plotted as mean values ±S.D. The thiazolidinediones inhibited cell proliferation of DRO cells in a dose responsive fashion (FIG. 3B). The calculated inhibitory concentration at which 50% of cell proliferation was inhibited (IC50) was about 0.8 nM for RS5444, about 75 nM for rosiglitazone, and about 1412 nM for troglitazone (FIG. 3B). The EC50 concentrations were in close agreement with the IC50 concentrations for each compound (FIG. 3B). These results suggest that all three thiazolidinediones act through PPARγ for their growth inhibitory effects on DRO cells in culture.

RS5444 specifically activates PPARγ, but not PPARα or PPARδ. The rat small intestinal cell line, RIE, which does not express PPARs, was treated with 10 nM RS5444 following transient transfection with the appropriate PPAR isoform (γ, α or δ) and PPAR response element linked to a luciferase reporter. Increased luciferase activity was only observed in the presence of PPARγ and PPRE3-tk-luc.

The ability of RS5444 to inhibit clonogenic growth of DRO cells in soft agar was also examined (FIG. 3C). DRO cells were plated in triplicate on soft agar for colony formation and treated with the indicated doses of RS5444. Colonies were stained and counted after two weeks. Inhibition was dose responsive, with inhibition of 63% at 1 nM, 98% at 10 nM and 99.4% at 100 nM RS5444 (FIG. 3C).

RS5444 also was observed to inhibit ATC tumor growth in athymic nude mice in a dose responsive manner. RS5444 was added to the rodent diet one week prior to tumor implantation with DRO. Tumor growth was inhibited by 89.7% with 0.025% RS5444 and by 54.5% with 0.0025% RS5444. No inhibition of tumor growth was observed with 0.00025% RS5444. In the 0.025% diet group, 5 of 10 mice did not form tumors.

These results demonstrate that PPARγ and RXR polypeptides are expressed in human ATC tissues and are functional as transcriptional factors in human ATC cells in response to PPARγ agonists. In addition, these results demonstrate that RS5444 activates PPARγ at sub-nanomolar concentrations, and that these same concentrations of RS5444 inhibit cell proliferation and tumor growth in human ATC cells.

### Example 2

### RhoB is Upregulated by PPARγ Agonists

A PPARγ agonist activates PPARγ to elevate p21^{WAF1/CIP1} polypeptide levels in ATC cells, and elevation of p21^{WAF1/CIP1} levels are required for the growth inhibitory effects of a PPARγ agonist in human ATC cells, as described elsewhere (Copland et al., Oncogene, 25(16):2304-17 (2006)).

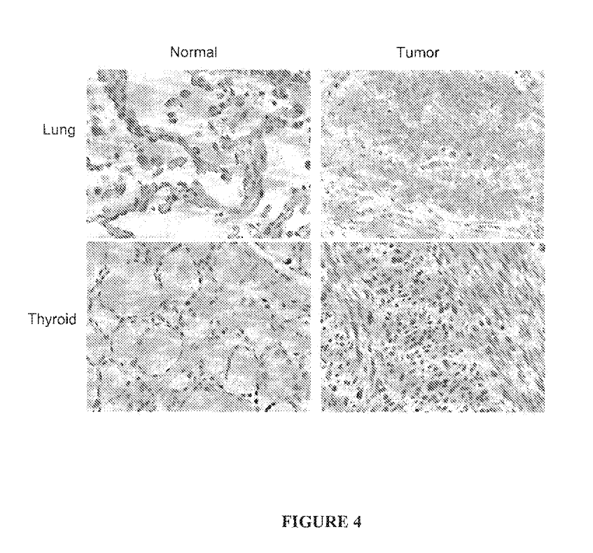

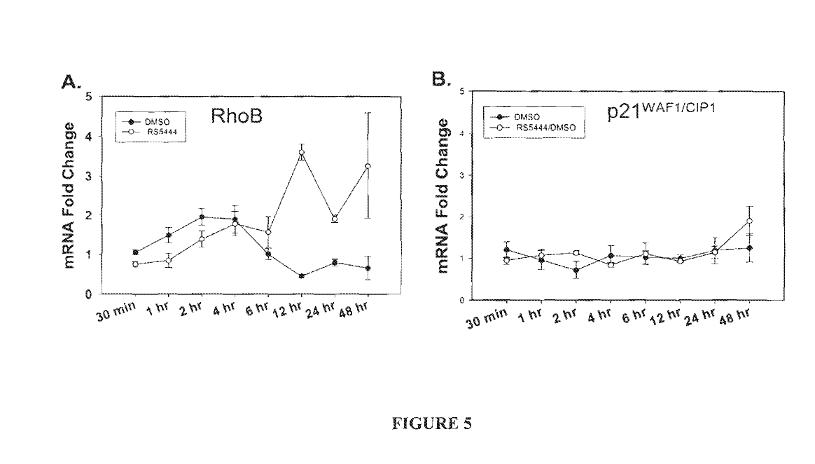

Immunohistochemistry was used to analyze RhoB polypeptide expression in formalin-fixed, paraffin-embedded patient tissues with the polyclonal antibody (C5, Santa Cruz, 1:200 dilution). RhoB was expressed in normal thyrocytes and appeared to be expressed in ATC patient tissues (FIG. 4). Additional matched normal thyroid and ATC tissues are being examined to determine whether RhoB levels are attenuated in ATC tissues, as observed in lung cancer, since loss of RhoB polypeptide expression correlates with poorly differentiated tumors (lung, brain, head and neck cancers) and poor patient prognosis (Forget et al., Clin Exp Metastasis, 19:9-15 (2002); Mazieres et al., Clin Cancer Res, 10:2742-2750 (2004); and Adnane et al., Clin Cancer Res, 8:2225-2232 (2002)). RhoB was expressed predominantly in the nucleus of normal thyroid cells and ATC tissues (FIG. 4, Thyroid normal and Thyroid Tumor panels). In contrast, predominantly cytoplasmic/membrane expression was observed in lung tissue (FIG. 4, Lung Normal and Tumor panels).

Experiments also were conducted to examine the expression of RhoB in response to treatment with a PPARγ agonist. Cells from the human ATC cell line, DRO, were treated at various time points during 48 hours with DMSO control (0.1%) or 10 nM RS5444. RhoB and p21^{WAF1/CIP1} RNA levels were measured using real-time PCR. Data were normalized to DMSO untreated controls for each time point measured. RhoB mRNA levels were significantly elevated at 12 hours through 48 hours (FIG. 5A), while no change in p21^{WAF1/CIP1} RNA was observed at any time point measured (FIG. 5B).

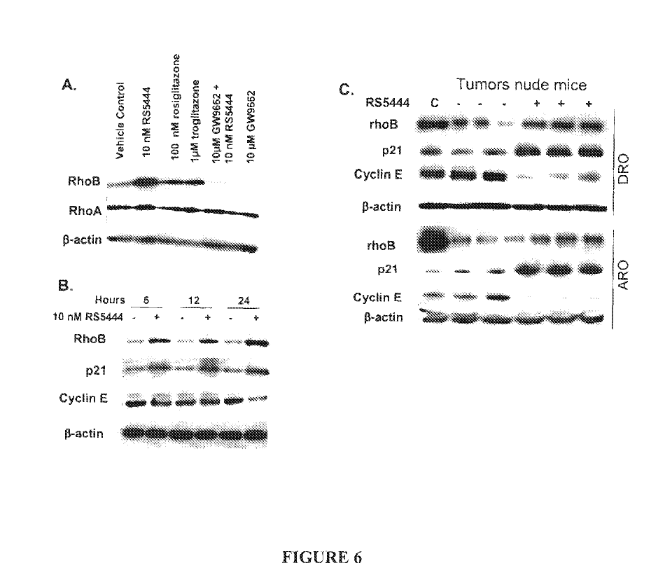

RhoB and p21^{WAF1/CIP1} polypeptide expression also was examined in ATC cells to determine the time course of induction of each polypeptide following stimulation with a PPARγ agonist (FIG. 6). DRO cells were treated with 10 nM RS5444, 100 nM rosiglitazone, 1 μM troglitazone, the PPARγ antagonist GW9662 alone or in combination with 10 nM RS5444, or with vehicle control (0.1% DMSO). Polypeptide lysates were prepared and examined for RhoB, RhoA and β-actin expression. The three thiazolidinediones (RS5444, rosiglitazone, and troglitazone) induced RhoB polypeptide levels 24 hours after a single treatment, as compared to vehicle control (0.1% DMSO; FIG. 6A). Treatment with the PPARγ antagonist, GW9662, blocked the ability of RS5444 to elevate RhoB polypeptide expression. These results demonstrated that induction of RhoB is dependent upon activation of PPARγ (FIG. 6A, compare last two lanes to second lane).

The time course for induction of RhoB and p21^{WAF1/CIP1} was determined using Western blotting. Levels of both polypeptides were significantly elevated at 6 hours following treatment with 10 nM RS5444 (+) compared to respective vehicle controls (−), and RhoB and p21^{WAF1/CIP1} remained elevated for 24 hours (FIG. 6B). Cyclin E levels were down-regulated in response to RS5444 in DRO cells at 24 hours, but not at earlier time points measured (FIG. 6B).

RhoB and p21^{WAF1/CIP1} polypeptide expression also was examined in DRO and ARO tumor tissues removed from athymic nude mice following 35 days of treatment with vehicle control (−) or 0.025% RS5444 in the diet. Beta-actin was used as a normalization control to ensure even loading of total polypeptide in all lanes. RhoB and p21^{WAF1/CIP1} polypeptide expression was elevated while cyclin E levels were down-regulated in DRO and ARO ATC tumors exposed to 0.025% RS5444 for 35 days (FIG. 6C). Collectively, the data demonstrate that RhoB and p21^{WAF1/CIP1} polypeptides are elevated rapidly upon exposure to a PPARγ agonist and remain elevated with chronic treatment of a PPARγ agonist.

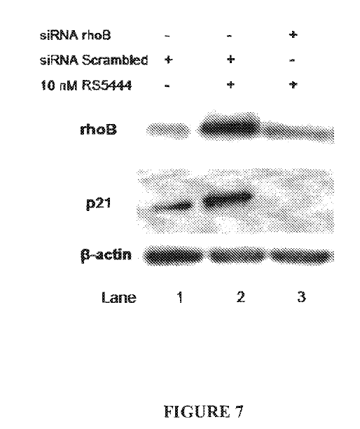

RhoB was shown to be essential for elevating p21^{WAF1/CIP1} polypeptide levels (FIG. 7). DRO cells were transfected with siRNA against RhoB or scrambled control siRNA. After 24 hours, the cells were treated with 10 nM RS5444, and cell lysates were prepared 24 hours after RS5444 stimulation. RhoB, p21^{WAF1/CIP1} and β-actin polypeptides were analyzed by Western blotting. RhoB polypeptide expression was silenced using siRNA against RhoB (FIG. 7, top row, lane 2 versus 3). Expression of p21^{WAF1/CIP1} polypeptide was not elevated in RS5444-stimulated cells in which expression of RhoB was silenced (FIG. 7, middle row, lane 2 versus 3). RhoB expression was, therefore, necessary for p21^{WAF1/CIP1} expression.

These results indicate that RhoB is expressed in normal thyroid tissues. Expression of RhoB appears to be attenuated in human ATC tissues, similar to that in poorly differentiated lung cancer. RhoB is upregulated at the RNA and polypeptide level after treatment with a PPARγ agonist. This upregulation of RhoB expression is early, within hours, and remains elevated for at least two days. ATC tumors in animals chronically exposed to a PPARγ agonist demonstrate elevated RhoB polypeptide levels. The p21^{WAF1/CIP1} polypeptide level is elevated in a manner similar to that of RhoB in response to a PPARγ agonist. However, p21^{WAF1/CIP1} levels are regulated at the polypeptide level and require the presence of RhoB. In the absence of RhoB, p21^{WAF1/CIP1} polypeptide levels are not elevated by a PPARγ agonist.

### Example 3

### RhoB and p21^{WAF1/CIP1} Co-Localize to the Nucleus in Cells Stimulated with a PPARγ Agonist

Typically, RhoB resides in endosomes to regulate trafficking of cell surface membrane polypeptides into the cell, while localization of p21^{WAF1/CIP1} is nuclear to regulate cell cycle kinetics.

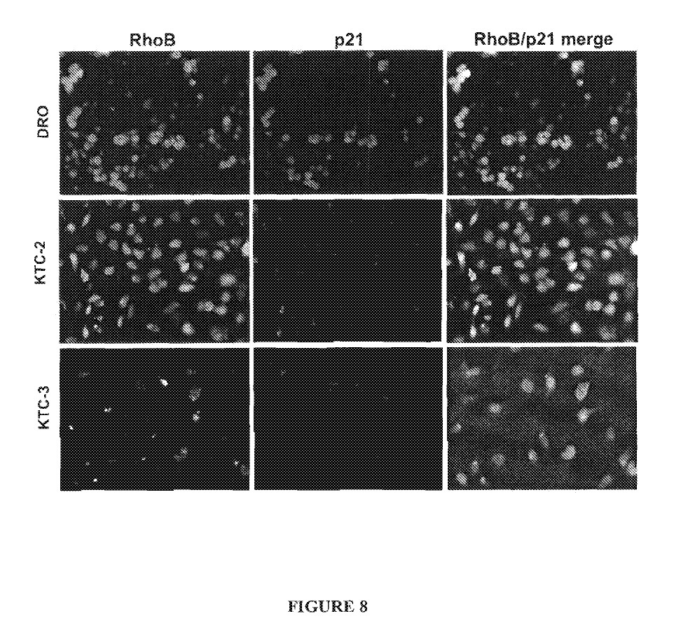

Experiments were performed to examine the cellular localization of RhoB and p21^{WAF1/CIP1} in three different human ATC cell lines (DRO, KTC-2, KTC-3). DRO, KTC-2, and KTC-3 cells were treated for 24 hours with 10 nM RS5444 and examined for RhoB and p21^{WAF1/CIP1} expression by immunocytochemistry (ICC). Fluorescently labeled antibodies were used to detect RhoB (FITC, green) and p21^{WAF1/CIP1} (Texas red). Immunofluorescent photographs were taken at 60× magnification. RhoB appeared predominantly in the nucleus, co-localized with p21^{WAF1/CIP1} (FIG. 8).

Collectively, these data and the IHC data for RhoB (FIG. 4) demonstrate that RhoB can be detected in the nucleus, and that a PPARγ agonist upregulates RhoB polypeptide levels in human ATC cells.

### Example 4

### Identification of Nucleic Acids Regulated by PPARγ

Affymetrix Plus 2.0 arrays were used to identify nucleic acids altered between control vehicle-treated and RS5444-treated ATC cells from cell culture and tumors implanted ectopically into athymic nude mice. Cells in culture were treated with a single dose of 10 nM RS5444 for either 2, 6, or 24 hours. Animals were treated with 0.025% RS5444 after implantation of 1×10⁶ DRO cells. Animals were fed a diet including 0.025% RS5444 for 35 days, and tumor volume was measured weekly. Tumor tissues were removed, RNA was isolated, and nucleic acid expression was analyzed. Examples of some nucleic acids, the expression of which was altered in both the animal derived tumors and cells in culture, are presented in Table 1. Some of the nucleic acids regulated by PPARγ in ATC cells (e.g., BMP2, MMP14, ANGPTL4, and ITGA5) are secreted polypeptides (Table 1).

TABLE 1
Nucleic acids altered in response to RS5444 treatment of DRO cells in cell culture
and tumors derived from ATC tumors implanted into athymic nude mice.

| | | | | | |
|---|---|---|---|---|---|
| GenBank | Nucleic Acid | Nucleic Acid | Chromosome | Fold change | Fold change |
| Accession No. | Symbol | Name | location | in vitro | in vivo |

Upregulated Nucleic Acids

| | | | | | | |
|---|---|---|---|---|---|---|
| BI668074 | RhoB | ras homolog | chr2p24 | 3.4 | (2 h) | 2 |
| AI263909 | | gene family, | | 4.1 | (6 h) | |
| | | member B | | 2.8 | (24 h) | |
| AA583044 | BMP2 | Bone morphogenetic | chr20p12 | 2.5 | (24 h) | 3.8 |
| | | protein 2 | | | | |
| NM_016109 | ANGPTL4 | angiopoietin-like | chr19p13.3 | 5.3 | (2 h) | 2 |
| | | 4 | | 4.9 | (6 h) | |
| | | | | 8.4 | (24 h) | |
| X83535 | MMP14 | matrix | chr14q11-q12 | 5.4 | (2 h) | 2.5 |
| | | metalloproteinase | | 16.9 | (24 h) | |
| | | 14 (membrane- | | | | |
| | | inserted) | | | | |
| BF528433 | HR | hairless homolog | chr8p21.2 | 2 | (2 h) | 1.5 |
| AF039196 | | (mouse) | | 2.3 | (24 h) | |
| NM_002205 | ITGA5 | integrin, alpha 5 | chr12q11-q13 | 2.2 | (24 h) | 1.9 |
| | | (fibronectin | | | | |
| | | receptor, alpha | | | | |
| | | polypeptide) | | | | |
| M15330 | IL1B | interleukin 1, | chr2q14 | 14.2 | (24 h) | 5.6 |
| | | beta | | | | |

Downregulated nucleic acids

| | | | | | | |
|---|---|---|---|---|---|---|
| AI625747 | ADRB1 | adrenergic, beta- | chr10q24-q26 | −3.3 | (2 h) | −2 |
| | | 1-receptor | | −2.2 | (24 h) | |
| NM_005570 | LMAN1 | lectin, mannose- | chr18q21.3-q22 | −2 | (6 h) | −4.3 |
| | | binding, 1 | | | | |

### Example 5

### Elevation of p21^{WAF1/CIP1} Polypeptide in Response to a PPARγ Agonist is Required for Growth Inhibition

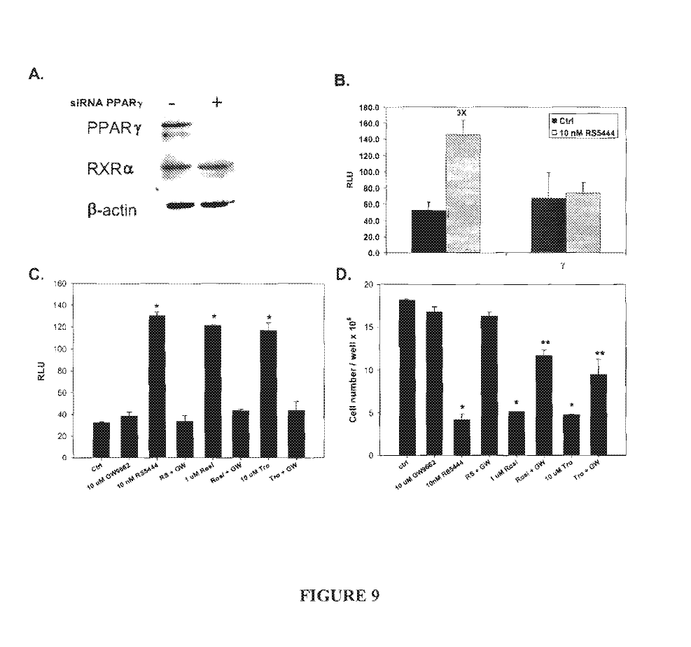

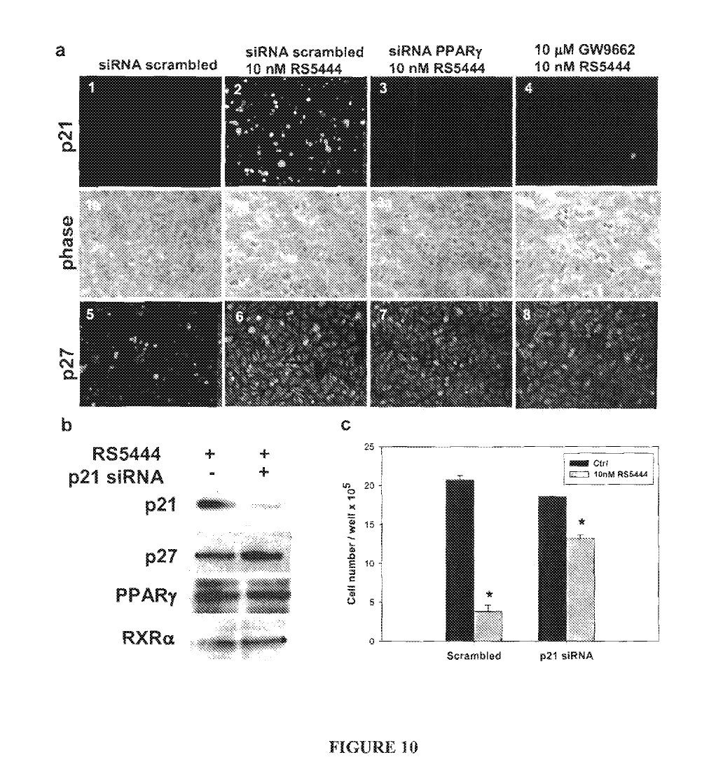

DRO cells were transfected with siRNA targeted to PPARγ, or with scrambled, control siRNA. Western blotting was performed 72 hours after treatment to analyze expression of PPARγ and RXRα polypeptides. Complete and selective inhibition of PPARγ polypeptide expression was observed (FIG. 9A).

Cells were transfected with siRNA for 72 hours followed by transfected with a PPRE3-tk-luc reporter and treatment with 10 nM RS5444 for 24 hours. PPRE3-tk-luc transfected cells (24 h) that were treated with 10 μM GW9662 followed by 10 nM RS5444 one hour later demonstrated complete loss of RS5444 inducible luciferase activity (FIG. 9B). Cotransfection with renilla luciferase allowed for normalization to relative light units (RLU). In PPRE3-tk-luc transfected DRO cells that were pretreated for 5 minutes with 10 μM GW9662 prior to treatment with 10 nM RS5444, 1 μM rosiglitazone, or 10 μM troglitazone, activation of PPARγ-induced luciferase activity was abolished (FIG. 9C).

DRO cells pretreated for one hour with 10 μM GW9662 prior to treatment with 10 nM RS5444, 1 μM rosiglitazone, or 10 μM troglitazone were no longer growth inhibited, demonstrating that PPARγ is essential for inhibition of cell proliferation by TZDs (FIG. 9D). DRO cells were treated with 10 μM GW9662, 10 nM RS5444, RS5444 and GW9662, 1 μM rosiglitazone, rosiglitazone and GW9662, 10 μM troglitazone, or troglitazone and GW9662 every 48 hours over six days. Cell numbers were counted and data were plotted as mean values ±S.D. (FIG. 9D).

These results indicate that functional PPARγ polypeptide is required for RS5444 mediated transcriptional activation (Copland et al., Oncogene, 25(16):2304-17 (2006)). In addition, functional PPARγ polypeptide is required for RS5444 mediated upregulation of p21^{WAF1/CIP1} polypeptide, and p21^{WAF1/CIP1} polypeptide mediates PPARγ inhibition of cell proliferation (FIG. 10; Copland et al., Oncogene, 25(16):2304-17 (2006)).

### Example 6

### Confirmation of RhoB as Therapeutic Response Biomarker

### Chemicals

PPAR-γ agonists RS5444 and troglitazone were obtained from Sankyo Company, Ltd. GW9662 and FTI-277 were obtained from Sigma-Aldrich (St. Louis, Mo.), and rosiglitazone was obtained from ChemPacific (Baltimore Md.). FK-228 or depsipeptide were obtained from Gloucester Pharmaceuticals, Inc. and the NCI.

### Cell Culture

DRO90-1 (DRO) and ARO81 (ARO) cells were derived from primary cultures of human anaplastic thyroid carcinoma tumors that were obtained from Dr. G. J. Juillard (University of California-Los Angeles, Los Angeles, Calif.), and KTC-2 and KTC3 cells were obtained from Dr. Junichi Kurebayashi (Podtcheko et al., J. Clin. Endocrinol. Metab., 88:1889-1896 (2003) and Pushkarev et al., Molecular mechanisms of the Effects of Low Concentrations of Taxol in Anaplastic Thyroid Cancer Cells. Endocrinology: en. 2004-0127 (2004)). Cells were cultured in RPMI 1640 medium (Cellgro, Herndon Va.) supplemented with 10% charcoal-stripped fetal bovine serum (Hyclone), non-essential amino acids, sodium pyruvate, and penicillin-streptomycin-amphotericin B at 37° C. in a humidified atmosphere with 5% CO². Charcoal-stripped serum was used to exclude endogenous PPAR ligands contained in serum. For proliferation studies, cells were plated in triplicate in 12-well culture plates at initial concentrations of 2×10⁴ cells/well. After overnight incubation, cells were treated with 10 nM RS5444 or DMSO diluent (Sigma) for 6 days with medium, and drug changed every 48 hours. After 6 days, cells were washed with PBS (Cellgro), trypsinized, and counted by Beckman Coulter Counter (Beckman Instruments, Fullerton Calif.). For morphology studies, cells were plated in 12 well plates at initial concentrations of 2.5×10⁴ cells/well. Cells were treated with either DMSO or 10 nM RS5444 (24 hours). After treatment, phase images were obtained on an inverted microscope. For real time PCR studies, cells were plated in 60 mm plates at 50% confluence and treated with 10 nM RS5444 for indicated incubation periods. For immunoblotting analyses, cells were plated in 6 cm plates at initial concentrations of 6×10⁵ cells/plate and were treated with either 10 nM RS5444, 10 μM rosiglitazone, 10 μM troglitazone, 10 μM GW9662, 1 ng/mL FK-228, 10 μM FTI-277 or indicated combinations for indicated incubation periods.

### Plasmids

pcDNA3.1 was obtained from Invitrogen, and pcDNA3.1-dominant negative RhoB was obtained from Séverine Steuve (Free University of Brussels). For transient transfection, cells were plated in 60 mm plates and transfected using Lipofectamine 2000 (Invitrogen, Carlsbad Calif.) for 24 hours followed by additional 24 hours after which cells were treated with 10 nM RS5444 or DMSO diluent. Cells were then collected, and mRNA isolated using RNAqueous (Ambion, Austin Tex.).

### Lentiviral Infection

Lentiviral constructs for the packaging of self-inactivating lentiviruses were made in the pLKO.1 vector (Sigma). The target sequence for PPAR-γ was 5′-GACAACAGACAAATCACCATT-3′, and a random scrambled sequence was used for the non-target vector (Sigma). Lentiviruses were produced by transient transfection in 293FT cells (Invitrogen) using ViraPower (Invitrogen) and Lipofectamine 2000 (Invitrogen) as per manufacturer's protocol. After 72 hours, the media was collected, sedimented, and filtered through a 0.45 μm polysulfonic syringe filter. Target ATC cells were plated in 10 cm plates and grown to 70% confluence for infection of lentivirus along with 5 μg/mL polybrene per manufacturer's protocol. After 48 hours, 2 μg/mL puramycin (Sigma) was added for selection of infected cells expressing shRNA.

siRNA Transfection

Cells were either plated in triplicate at 7.5×10⁴ cells/mL in 6-well plates or 2×10⁴ cells/well in 12-well plates, allowed to adhere overnight, and then transfected for 24 to 72 hours with 2 to 5 μg of either scrambled, p21^{WAF1/CIP1} (1022539), RhoB (SI00058933) or PPAR-γ-specific siRNA duplexes (Qiagen Valencia, Calif.) using RNAiFect reagent (Qiagen). The DNA target sequence for PPAR-γ was 5′-GCCCTTCACTACTGTTGAC-3′. After transfection, cells in the 6-well plates were treated with either DMSO diluent or 10 nM RS5444 for 24 hours and then analyzed by either real time PCR or immunoblotting. Cells in the 12-well plates were exposed for 6 days with medium, and drug changed every 48 hours. After 6 days, cells were washed with PBS (Cellgro), trypsinized, and counted by Beckman Coulter Counter.

### Luciferase Reporter Gene Analysis

Specific activation of PPRE3-tk-luc (R. Evans, Salk Institute, La Jolla, Calif.) by RS5444, rosiglitazone, and troglitazone was demonstrated by dual luciferase assay. Cells were plated in 6-well culture plates at initial concentrations of 2×10⁵ cells/well. After overnight incubation, cells were transfected with PPRE/luc and CMV/renilla luciferase plasmid using Lipofectamine 2000 (Invitrogen), and after 24 hours, cells were treated with PPAR-γ agonists at indicated concentrations. After 24 hours of drug treatments, cells were washed with PBS and lysed in 1× reporter lysis buffer (Roche), and luciferase activity was measured with a Dual Luciferase assay kit (Promega, Madison Wis.) with a luminometer (Veritas/Turner Biosystems). The enzyme activity was normalized for efficiency of transfection on the basis of renilla activity levels and reported as relative light units (RLU).

### Xenograft Studies

Suspensions of 1×10⁶/0.1 mL DRO or ARO cells in RPMI medium were injected subcutaneously in one flank of 3 to 4 week old athymic female nude mice. Mice were changed to specialized diets 1 week after tumor implantation and randomly assigned to diets consisting of either 0.025% RS5444 or sham control (Research Diets). Mice weighed between 20-25 grams and consumed on average 4 grams of food per day (1 mg/day of RS5444). Tumors were measured every 3 to 4 days for 35 days with calipers and tumor volumes were calculated by the formula: 0.5236(a×b×c), where a is the shortest diameter, b is the diameter perpendicular to a, and c is the diameter height.

### Cell Lysis and Immunoblotting

Cells were collected and lysed in RIPA buffer containing 50 mM Tris, 5 mM EDTA, 150 mM NaCl, 0.1% SDS, 0.5% Deoxycholate, 1% NP40, and protease inhibitor cocktail (Roche) followed by centrifugation. Excised tumor tissues were lysed in 50 mM Tris pH 8.0+1% SDS. Collected supernatants were, analyzed for protein concentration using Pierce's BCA assay, with 25 μg protein loaded per gel lane (Invitrogen) with later transfer to Immobilon-P membranes. The membranes were hybridized with antibodies (Santa Cruz, Cell Signaling, Sigma-Aldrich, St. Louis Mo.) as indicated overnight at 4° C. followed by treatment with species-specific horseradish peroxidase IgG (Jackson labs) for 45 minutes at room temperature. Detection was performed using Supersignal West Pico chemoluminescence kit (Pierce). Quantification of bands was performed in triplicate using Image Quant 5.2 (Molecular Dynamics, Sunnyvale Calif.).

### RNA Isolation, Real Time Quantitative PCR and RT-PCR

Total mRNA was isolated from cells using RNAqueous (Ambion) per the manufacturer's protocol followed by ethanol precipitation. Total RNA from normal thyroid tissue was extracted using TOTALLY RNA (Ambion) according to manufacturer's protocol followed by Chroma Spin Column 100 (BD Bioscience). Two-step quantitative reverse transcriptase-mediated real-time PCR (QPCR) was used to measure changes in mRNA levels of target genes in ATC cells. Total RNA was reverse transcribed using the High-Capacity cDNA Archive kit (Applied Biosystems) per the manufacturer's instructions. Twenty nanograms of cDNA were used as template in the subsequent QPCR reactions. Applied Biosystems' assays-on-demand assay mix of primers and TaqMan® MGB probes (FAMTM dye-labeled) for RhoB (Hs00269660_s1), RhoA (Hs00357608_m1), RhoC (Hs00237129_m1), Rac1 (Hs00251654_m1), cdc42 (Hs00741586_mH), p21 WAF1/CIP1 (CDKN1A Hs00355782_m1), and 18S rRNA (Hs99999901_s1) were used for QPCR measurements. Forty amplification cycles were performed on the Applied Biosystems Prism 7900 sequence detector, with cycle threshold (Ct) values calculated using the Sequence Detection System software (SDS 2.2.2. Applied Biosystems) normalized against 18S rRNA levels. Fold change values either between treated (RS5444) and control samples were calculated using the ΔΔCt method (Livak and Schmittgen, Methods, 25:402-408 (2001)).

For RT-PCR, cDNA was synthesized from 1 μg RNA using reverse transcriptase (Biorad) followed by PCR using the ThermalACE kit (Invitrogen) according to manufacturer's protocol. The primers used to amplify thyroglobulin (TG), thyroid-stimulating hormone receptor (TSH-R), sodium iodide symporter (NIS), PAX8, thyroid transcription factor 1 (TTF1), and glyceraldehyde-3-phosphate-dehydrogenase (GAPDH; as an internal control) are listed in Table 2. Reverse transcription (RT) conditions were 25° C. for 5 minutes, 42° C. for 30 minutes, and 85° C. for 5 minutes. PCR conditions were denaturation at 95° C. for 3 minutes, annealing at indicated temperatures for 30 seconds, primer extension at 74° C. for 1 minute, and then final extension for 10 minutes. PCR products were electrophoresed in 2% agarose gels, stained with ethidium bromide, and checked for the expected lengths.

TABLE 2
RT-PCR primers.

| Primers | Anneal | Cycles | bp | Sequence | |
|---|---|---|---|---|---|
| TG | 58° C. | 35 | 663 | Sense | 5′-GTT GGC AAC CTC ATC GT-3′ |
| | | | | Antisense | 5′-AAT TCT GCA GTG CCT GGT-3′ |
| TSH-R | 58° C. | 35 | 287 | Sense | 5′-GAA CTG ATA GCA AGA AAC ACC TGG-3′ |
| | | | | Antisense | 5′-GTA TCC TGG AAC TTG GAC TTT T-3′ |
| NIS | 60° C. | 40 | 234 | Sense | 5′-TCC ATG TAT GGC GTG AAC C-3′ |
| | | | | Antisense | 5′-CTT CGA AGA TGT CCA GCA CC-3′ |
| PAX8 | 58° C. | 35 | 155 | Sense | 5′-TAC TCT GGC AAT GCC TAT GG-3′ |
| | | | | Antisense | 5′-TAC AGA TGG TCA AAG GCC G-3′ |
| PPARγ | 56° C. | 35 | 360 | Sense | 5′-TCT GGC CCA CCA ACT TTG GG-3′ |
| | | | | Antisense | 5′-CTT CAC AAG CAT GAA CTC CA-3′ |
| TTF-1 | 54° C. | 35 | 268 | Sense | 5′-AAC CTG GGC AAC ATG AGC-3′ |
| | | | | Antisense | 5′-GTC GCT CCA GCT CGT ACA C-3′ |
| GAPDH | 60° C. | 35 | 450 | Sense | 5′-ACC ACA GTC CAT GCC ATC AC-3′ |
| | | | | Antisense | 5′-TCC ACC ACC CTG TTG CTG TA-3′ |

### Statistical Analysis

Data are presented as the mean ±SD and comparisons of treatment groups were analyzed by 2-tailed paired Student's t test. Data for comparison of multiple groups were presented as mean ±SD and were analyzed by 2-way ANOVA. p<0.05 was considered statistically significant.

### Results

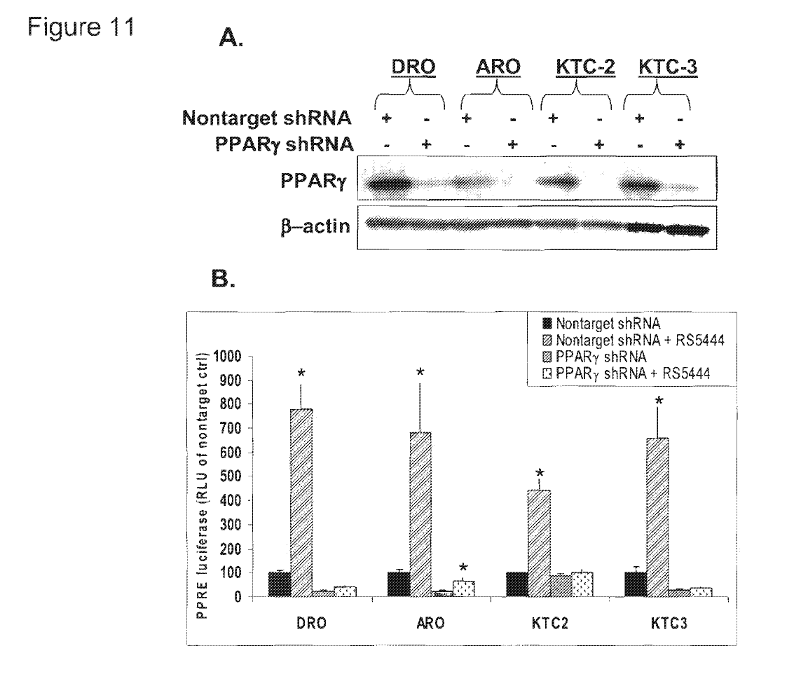

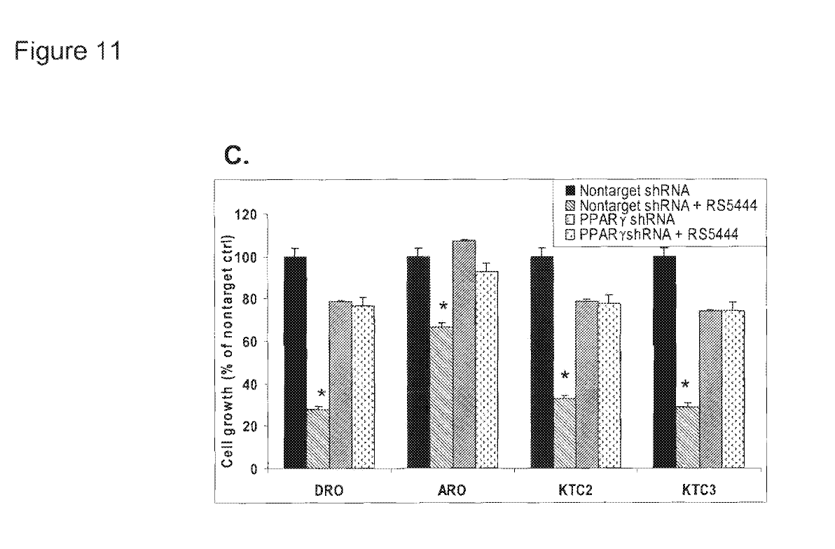

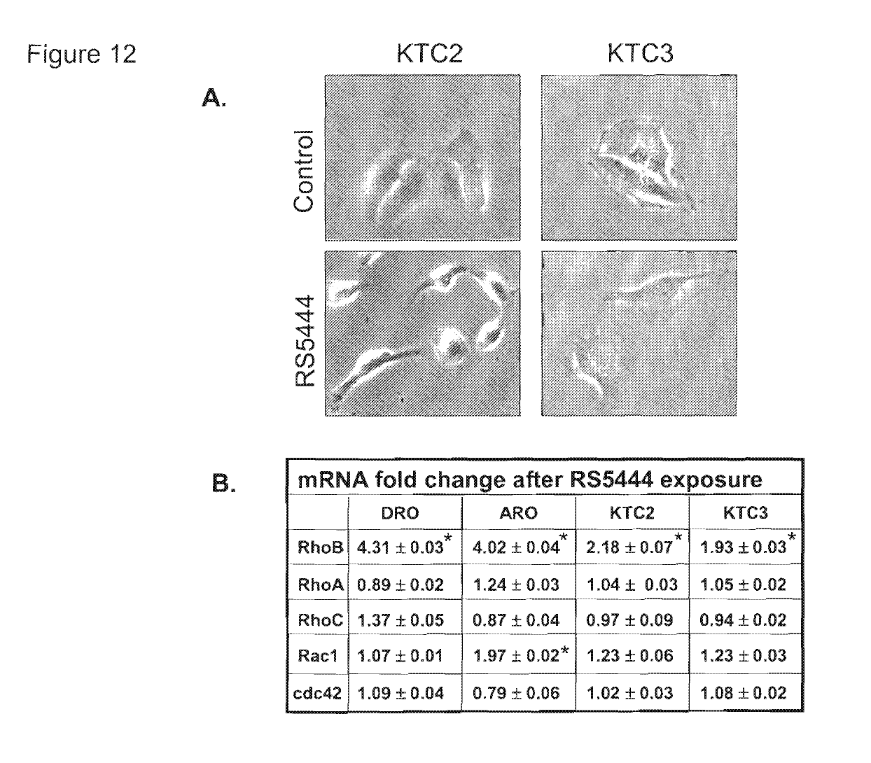

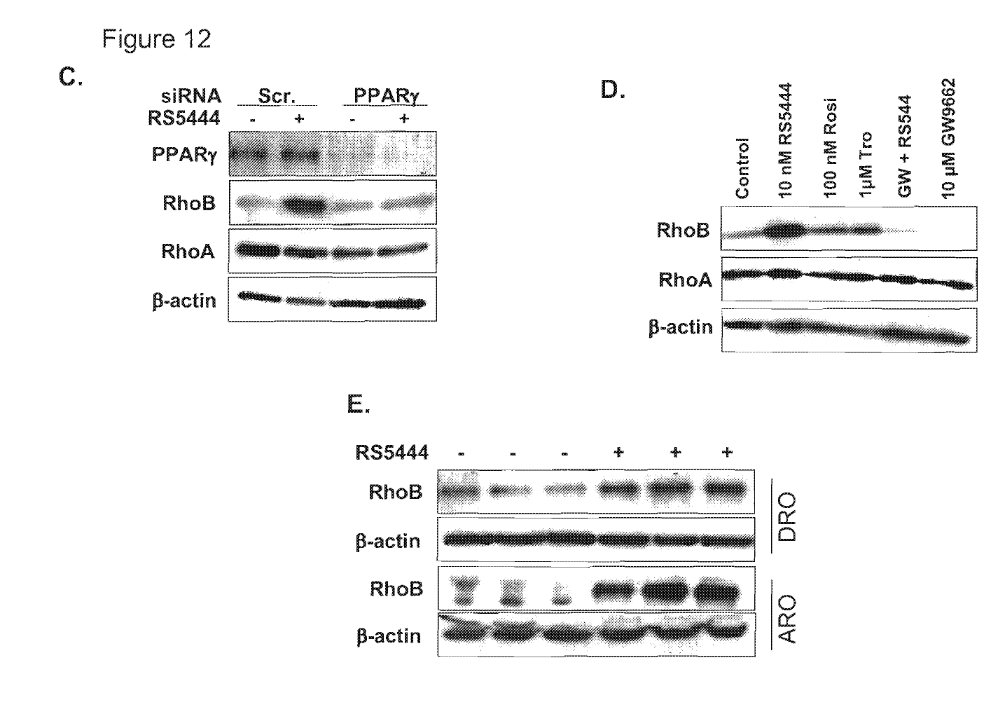

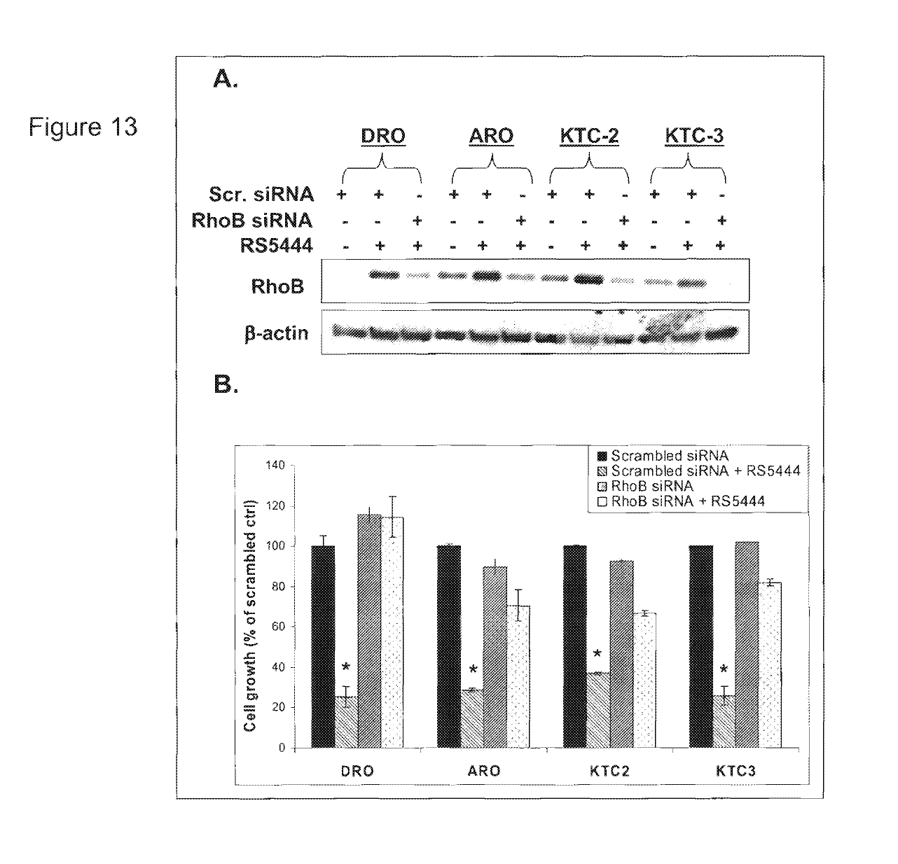

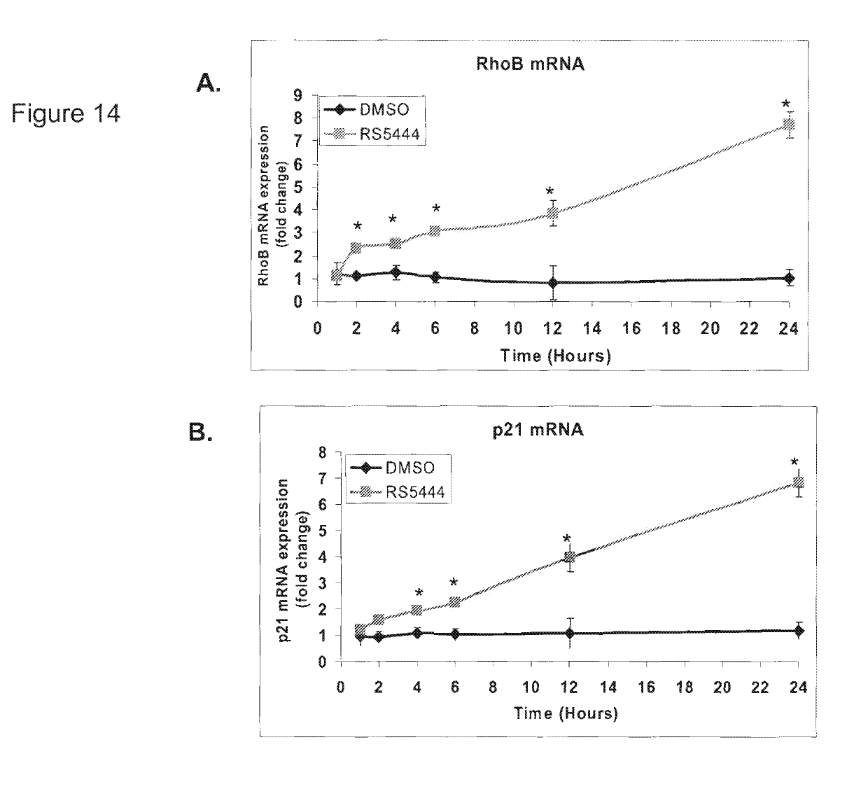

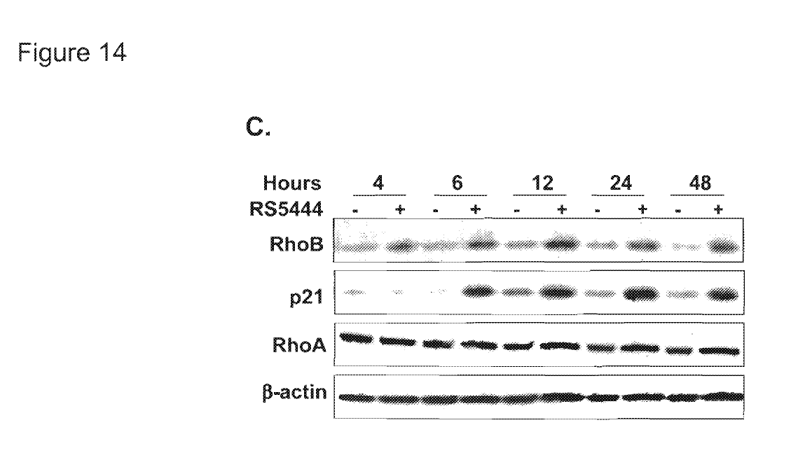

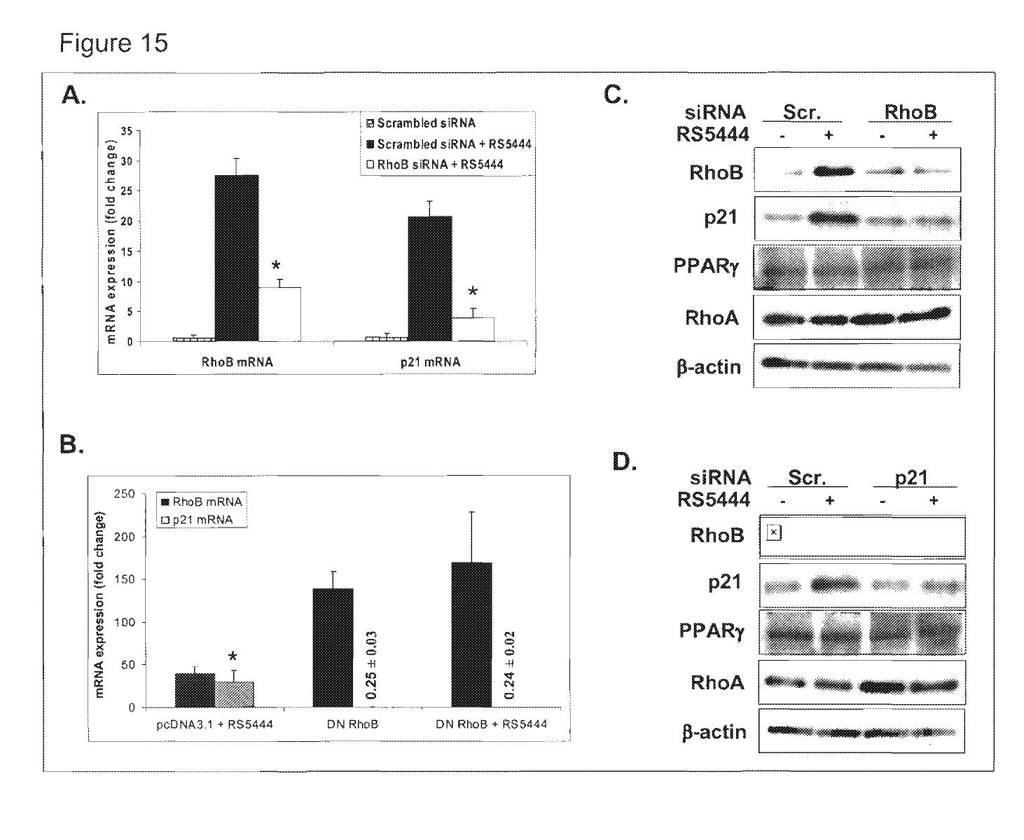

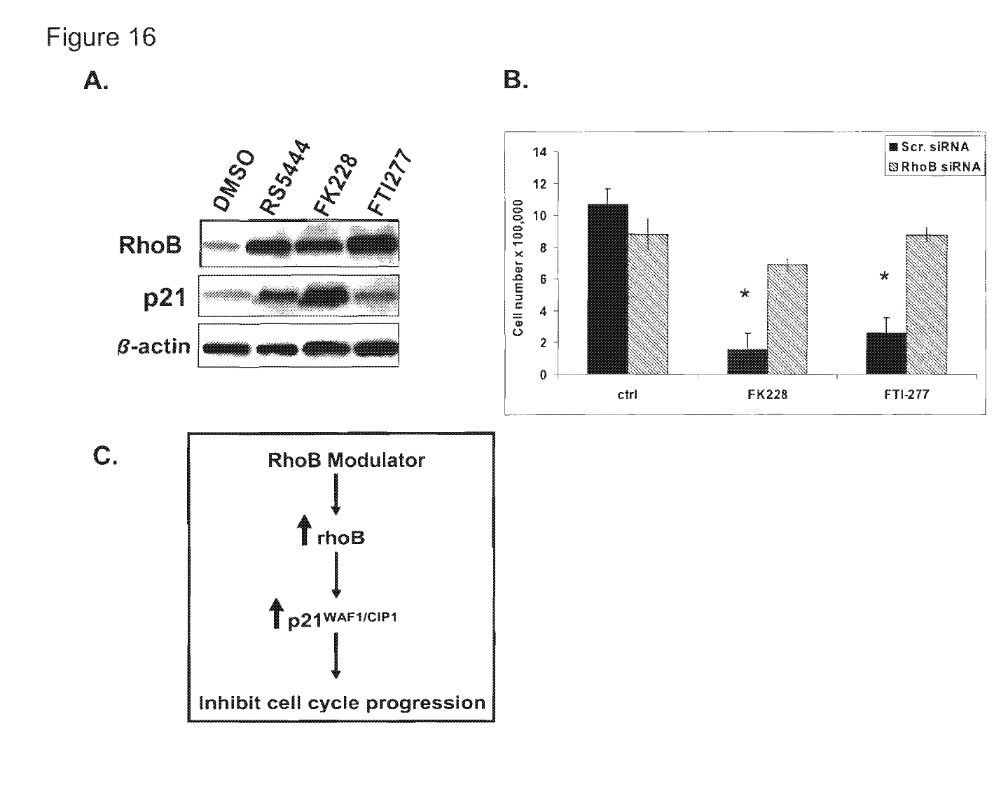

The results presented in FIGS. 11-16 confirm that RhoB can be used as therapeutic response biomarker.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method for assessing the effectiveness of a cancer treatment in a mammal, said method comprising (a) determining whether or not said mammal has a therapeutic response profile, wherein said mammal has received said treatment, and (b) classifying said treatment as effective if said mammal has said therapeutic response profile and classifying said as not being effective if said mammal does not have said therapeutic response profile.

2. The method of claim 1, wherein said mammal is a human.

3. The method of claim 1, wherein said treatment is treatment with a PPARγ agonist.

4. The method of claim 3, wherein said PPARγ agonist is rosiglitazone or troglitazone.

5. The method of claim 1, wherein said profile is determined in thyroid tissue.

6. The method of claim 5, wherein said thyroid tissue is biopsy tissue.

7. The method of claim 6, wherein said biopsy tissue is obtained within 24 hours of said treatment.

8. The method of claim 1, wherein said profile is determined using PCR or a nucleic acid array.

9. The method of claim 1, wherein said profile is determined using immunohistochemistry or an array for detecting polypeptides.

10. A method for assessing the effectiveness of a cancer treatment in a mammal, said method comprising (a) determining whether or not a mammal having cancer and having received a cancer treatment comprises RhoB nucleic acid or polypeptide at a level that differs from the average level in mammals having cancer and not having received a cancer treatment, and (b) classifying said treatment as effective if said level differs from said average level and classifying said treatment as ineffective if said level does not differ from said average level.

11. The method of claim 10, wherein said mammal is a human.

12. The method of claim 10, wherein said treatment is treatment with a PPARγ agonist.

13. The method of claim 12, wherein said PPARγ agonist is rosiglitazone or troglitazone.

14. A method for assessing the effectiveness of a cancer treatment in a mammal, said method comprising (a) determining whether or not a mammal having cancer and having received a cancer treatment comprises a BMP2, MMP14, ANGPTL4, or ITGA5 nucleic acid or polypeptide at a level that differs from the average level in mammals having cancer and not having received a cancer treatment, and (b) classifying said treatment as effective if said level differs from said average level and classifying said treatment as ineffective if said level does not differ from said average level.

15. The method of claim 14, wherein said level is determined in blood.

16. The method of claim 14, wherein said mammal is a human.

17. The method of claim 14, wherein said treatment is treatment with a PPARγ agonist.

18. The method of claim 17, wherein said PPARγ agonist is rosiglitazone or troglitazone.
